(19) United States
(12) Patent Application Publication
(10) Pub. No.: US 2010/0035802 A1
(43) Pub. Date: Feb. 11, 2010

(54) PARTICULAR SHORT-CHAIN PEPTIDE CANCER DRUG AND ITS TREATMENT

(76) Inventor: Fu-Hsin CHANG, Kaohsiung City (TW)

Correspondence Address: Wang Law Firm, Inc., 4989 Peachtree Parkway,, Suite 200, Norcross, GA, 30092, US

(21) Appl. No.: 11/849,323

(22) Filed: Sep. 3, 2007

(51) Int. Cl. A61K 38/16; C07K 14/00; A61P 35/00

(52) U.S. Cl. 514/12; 530/324

(57) ABSTRACT

The invention relates to a particular short-chain peptide cancer drug and its treatment. The cancer drug includes a short-chain peptide composed of amino acids interlinked in a unique sequence. The treatment is based on a cancer angiogenesis targeted therapy model, proceeded by an arterial injection of the short-chain peptide cancer drug, so that the short-chain peptide can enter and be amassed in cavities of new blood vessels of cancerous tissues, further inducing apoptosis of cancer cells to death.

### BACKGROUND OF THE INVENTION

1. Field of the Invention

This invention relates to a particular short-chain peptide cancer drug and its treatment, particularly to one utilizing a unique short-chain peptide to quickly identify and kill cancer cells through a certain cancer therapy model.

2. Description of the Prior Art

Commonly, there are three treatments employed to cure patients with cancer. They are surgery, radiotherapy and chemotherapy.

Surgery is usually used for treating cancer patients who are graded as stage II or below, with considerations such as age, condition of heart etc. Although it is a good therapy, but aftereffects are easy to be generated after surgery.

Radiotherapy is precisely called palliative radiotherapy, mainly employed to treat partial region of tumor by means of high-energy radioactivity emitted to kill cancer cells. However, during radiation, healthy cells are meanwhile destroyed to cause tremendous side effects.

Chemotherapy is to inject chemical drugs (cancer-inhibiting drugs) into a body by an intravenous drip, used for treating patients who had a surgical operation or whose cancer cells have been spreading too widely to be controllable. Like radiotherapy, chemotherapy can not only kill cancer cells but also damage healthy ones. Therefore, for example, a patient with kidney disease is not suitable to be treated by chemotherapy. In addition, chemotherapy is the most uncomfortable therapy as it would always cause side effects such as vomiting, hair loss and extreme pain etc.

As biotech has been developed vigorously in recent years, scientists have zealously been exploring to find other methods for cancer therapy. Among them, genetic therapy is believed to be the hopeful one, especially gene replacement that is carried out via replacing congenital defective genes with normal genes to achieve curing purpose. Actually, there are still a lot of puzzles to be broken through, such as the development of a more effective and safety carrier to transfer genes after recombination to target cells for replacement. In addition, the biggest challenge for genetic therapy is the safety of gene transplantation. Because genetic therapy may alter those that are inherent, gene transplantation must be carried out with autologous cells. And, transplantation of germ cell can not be done discretionarily. So, genetic therapy still has problems of safety and stability to be solved technically.

In addition, while cancer cells grow at the beginning stage, they or their surrounding connective tissues can excrete plenty of angiogenic substances able to activate the endothelial cells of blood vessels to be quickly recombined to form angiogenesis that can tremendously affect the growth and the spreading of tumors. As a tumor has its diameter increased with 2 mm, angiogenesis has to be carried out so as to gain nutrients from the outside to nourish cancer cells. However, new blood vessels are always created so swiftly that they are formed incompletely to have a porous vessel wall with cavities ranging from 60 to 100 Å, larger than those of healthy blood vessels. That is the basis that Anti-angiogenesis therapy model is derived from.

Anti-angiogenesis therapy model is a treatment that uses drugs to block blood vessels created around cancer cells to prevent cancer cells from nourished with matrix metalloproteinases, so as to stop cancerous tumors from developing new blood vessels. But, anti-angiogenesis therapy can only effectively be carried out in case that the locations of tumors are definitely identified. And, as drugs are transferred by arterial injection, the creation of healthy blood vessels are to be affected somewhat. Therefore, anti-angiogenesis therapy is so far a targeted treatment, still under research, development and evaluation.

### SUMMARY OF THE INVENTION

The objective of this invention is to offer a short-chain peptide cancer drug treated by a cancer angiogenesis targeted therapy model to quickly identify and kill cancer cells under a stable and secure procedure, almost causing no side effects.

The present invention is characterized that the cancer drug includes a short-chain peptide composed of a unique sequence of amino acids, which is treated by the cancer angiogenesis targeted therapy model, able to quickly identify the locations of cancerous tissues and induce apoptosis of cancer cells to the death, so as to achieve the purpose of cancer therapy.

A various proteins with different chain lengths are selected in the present invention for being trimmed into segments with diverse lengths, which are diversely combined to become numerous peptides with different sequences. Via numerous experiments and comparisons, a unique short-chained peptide is selected, with a sequence of

[symbol]

R-G-D-L-A-S-L-Q-K-A-A-H-G-H-V-R-K-A-F-K-S-H-V-S-
T-L-T-D-L-Q-P-Y-M-R-Q-F-V-A-H-L-Q-E-T-S-P-L-R-D

[symbol]

that is composed of 48 amino acids. The short-chain peptide is the essential protein for cell cleavage. Cells can not proceed with normal cleavage if a lack of the protein. It has been experimentally found that when cancer cells are added with the short-chain peptide of the present invention, they can not create proteins necessary for their normal cleavage, but stagnating on the stage of cell cleavage for being further induced to apoptosis to the death. So, the short-chain peptide of the invention really possesses an extreme potential to kill cancer cells.

The cancer angiogenesis targeted therapy model used in the present invention is treated by an arterial injection of the cancer drug containing the short-chain peptide. Having a size larger than cavities of blood capillaries and smaller than those of new blood vessels, the short-chain peptides are to automatically enter and be amassed in the cavities of new blood vessels to induce apoptosis of cancer cells to the death.

### BRIEF DESCRIPTION OF DRAWINGS

This invention is better understood by referring to the accompanying drawings, wherein:

FIG. 1 is a graph showing the survival rates of cancer cells without being added with the particular short-chain peptide in the present invention; and

FIG. 2 is a graph showing the survival rates of cancer cells added with the particular short-chain peptide in the present invention.

### DETAILED DESCRIPTION OF THE PREFERRED EMBODIMENT

A preferred embodiment of a particular short-chain peptide cancer drug and its treatment in the present invention is characterized that the cancer drug includes a short-chain peptide having a unique sequence of amino acids sequence and the treatment is based on cancer angiogenesis targeted therapy model, which can quickly identify the locations of cancerous tissues and transmit the cancer drug containing the short-chain peptide thereof, so as to induce apoptosis of cancer cells to death.

A peptide is composed of numerous amino acids linked together to be a chained compound, having diverse biochemical functions depending on the type and the sequence of amino acids. A number of peptides are linked and folded up to form a variety of proteins. Therefore, peptides can be regarded as short-chained proteins.

A various proteins with different chain lengths are selected in the present invention for being trimmed into segments with diverse lengths, which are diversely combined to become numerous peptides with different sequences. Via numerous experiments and comparisons, a unique short-chained peptide is selected, with a sequence of

[symbol]

R-G-D-L-A-S-L-Q-K-A-A-H-G-H-V-R-K-A-F-K-S-H-V-S-
T-L-T-D-L-Q-P-Y-M-R-Q-F-V-A-H-L-Q-E-T-S-P-L-R-D

[symbol]

that is composed of 48 amino acids, which are Arginine, Glycine, Aspartic acid, Leucine, Alanine, Serine, Leucine, Glutamine, Lysine, Alanine, Alanine, Histidine, Glycine, Histidine, Valine, Arginine, Lysine, Alanine, Phenylalanine, Lysine, Serine, Histidine, Valine, Serine, Threonine, Leucine, Threonine, Aspartic acid, Leucine, Glutamine, Proline, Tyrosine, Methionine, Arginine, Glutamine, Phenylalanine, Valine, Alanine, Histidine, Leucine, Glutamine, Glutamic acid, Threonine, Serine, Proline, Leucine, Arginine and Aspartic acid in order. The short-chain peptide is the essential protein for cell cleavage. Cells can not proceed with normal cleavage if a lack of the protein. It has been experimentally found that as cancer cells are added with the short-chain peptide, they can not create proteins necessary for their normal cleavage, but stagnating on the stage of cell cleavage for being further induced to apoptosis to death. So, the short-chain peptide with the unique sequence really possesses an extreme potential to become an effective therapy for cancerous diseases.

The treatment of the particular short-chain peptide cancer drug in the present invention is based on the model and the concept of anti-angiogenesis to establish a unique cancer angiogenesis targeted therapy model, which is treated by an arterial injection of the cancer drug containing the short-chain peptide. Having a size larger than cavities of blood capillaries and smaller than those of new blood vessels, the short-chain peptides are to automatically enter and be amassed in the cavities of new blood vessels to gradually induce apoptosis of cancer cells until death. Such a therapy model is also available for polymeric drugs having the same curative effects for curing cancerous diseases.

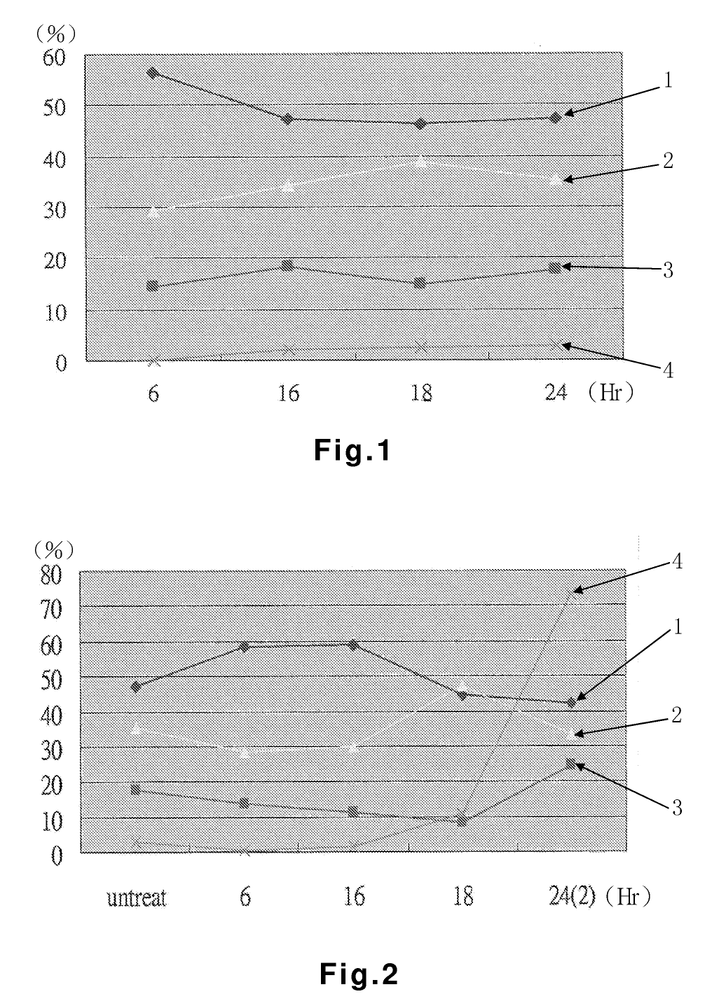

FIG. 1 shows the survival rate of cancer cells in cell experiments added with none of the particular short-chain peptide. FIG. 2 shows the survival rate of cancer cells in cell experiments added with the particular short-chain peptide. The percent of normal cancer cells 1 is equal to the number of normal cancer cells divided by the summation of the number of normal cancer cells, the number of interphase cancer cells and the number of mitotic phase cancer cells. The percent of interphase cancer cells 2 is equal to the number of interphase cancer cells divided by the summation of the number of normal cancer cells, the number of interphase cancer cells and the number of mitotic phase cancer cells. The percent of mitotic phase cancer cells 3 is equal to the number of mitotic phase cancer cells divided by the summation of the number of normal cancer cells, the number of interphase cancer cells and the number of mitotic phase cancer cells. The percent of dead cancer cells 4 is equal to the number of dead cancer cells divided by the summation of the number of normal cancer cells, the number of interphase cancer cells, the number of mitotic phase cancer cells and the number of dead cancer cells.

In cell experiments, the survival rate of cancer cells added with none of the short-chain peptide is as high as 95%. Apoptosis of cancer cells begins as soon as the short-chain peptide is added to cancer cells; it's obviously found out that after 24 hours, the survival rate of cancer cells is lowered to about 25% and the death rate is raised to about 70%, 35 to 40 times of normal death rate.

In Attachment, Photos 1˜3 show the cell cycles of liver cancer cells respectively added and not added with the particular short-chain peptide in cell experiments. Photos 4 and 5 show the growth of cancer cells respectively added and not added with the particular short-chain peptide in animal experiments.

As shown in Photo 1 of Attachment, the liver cancer cells are alive with a certain amount in cell experiments without being added with the short-chain peptide; they start to lessen quantitatively and carry out apoptosis, as shown in blue area, after being added with the short-chain peptide; and, as shown in Photo 3, after 24-hour culture, they die on a mass scale; after repeated experiments, it is found that the death rate of the liver cancer cells comes to about 70%.

And, as shown in Photo 4 of Attachment, the cancer cells have nuclei and a complete cell structure in the animal experiments, added with none of the short-chain peptide; and, as shown in Photo 5, after having been added with the short-chain peptide for a certain period of time, they die massively via apoptosis, with few cell bodies dieing of necrosis. With the experiments, it has been proved that the present invention can effectively kill cancer cells to lower their survival rate.

As the short-chain peptide of the present invention has a rather tiny size and can be metabolized, it can prevent a body from creating immune reaction that is apt to be caused by larger molecules such as proteins, after having it being injected into a human body. Moreover, it can carry out metabolism after a period of time, absorbed by the body without being accumulated therein to generate any influence. In addition, during the treatment of the cancer angiogenesis targeted therapy model of the present invention, the short-chain peptide of the present invention can selectively enter the cavities of blood vessels of cancerous tissues, instead of entering the vessel wall of blood capillaries to damage healthy cells, because of having a size bigger than the cavities of healthy blood vessels. So, the present invention is not only extremely effective for cancer therapy, but also more stable and securer than the conventional ones.

While the preferred embodiment of the invention has been described above, it will be recognized and understood that various modifications may be made therein and the appended claims are intended to cover all such modifications that may fall within the spirit and scope of the invention.

1. A particular short-chain peptide cancer drug characterized by comprising a particular short-chain peptide that includes a sequence of amino acids as [symbol]
Arg-Gly-Asp-Leu-Ala₅-Ser-Leu-Gln-Lys-Ala₁₀-
Ala-His-Gly-His-Val₁₅-Arg-Lys-Ala-Phe-Lys₂₀-Ser-
His-Val-Ser-Thr₂₅-Leu-Thr-Asp-Leu-Gln₃₀-Pro-Tyr-
Met-Arg-Gln₃₅-Phe-Val-Ala-His-Leu₄₀-Gln-Glu-Thr-
Ser-Pro₄₅-Leu-Arg-Asp [symbol]
said short-chain peptide able to induce apoptosis of cancer cells for cancer therapy.

2. A cancer therapy model characterized by an arterial injection of a cancer drug containing a particular short-chain peptide that has a size smaller than cavities of new blood vessels of cancerous tissues so as to be able to automatically enter and amass at said cavities of said new blood vessels of said cancerous tissues to induce apoptosis of said cancer cells to death.

3. A particular short-chain peptide cancer drug as claimed in claim 1, wherein said short-chain peptide is smaller than 60 Å.


SEQUENCE LISTING (1 sequence)

SEQ ID NO: 1 - protein, 48 aa, Artificial Sequence
  truncated protein fragments
RGDLASLQKAAHGHVRKAFKSHVSTLTDLQPYMRQFVAHLQETSPLRD